(19) United States
(12) Patent Application Publication
(10) Pub. No.: US 2006/0271010 A1
(43) Pub. Date: Nov. 30, 2006

(54) Loincloth diaper

(76) Inventor: Gary Dean LaVon, Liberty Township, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/135,689

(22) Filed: May 24, 2005

(51) Int. Cl. A61F 13/15

(52) U.S. Cl. 604/385.101; 604/385.26

(57) ABSTRACT

A disposable absorbent article includes a chassis and an absorbent assembly and is adapted to be configured as a loincloth diaper for use in combination with a support element that is worn around the waist of the wearer. The chassis has fastening elements for use in forming laterally extending tunnels adjacent to the waist edges through which the support element extends. The chassis includes a water-impermeable backsheet that may be folded laterally inward at both of its side edges to form opposing side flaps. Each side flap is attached to the interior surface of the chassis adjacent to its end edges. Each side flap has a longitudinally extending elastic gathering member attached adjacent to its proximal edge. The chassis may include an extensible formed web material. The absorbent assembly may be attached in a cruciform pattern to the chassis to allow unattached portions of the chassis to extend laterally.

### FIELD OF THE INVENTION

This invention relates to disposable absorbent articles such as disposable diapers and other articles intended for use on incontinent persons.

### BACKGROUND OF THE INVENTION

Disposable absorbent articles are designed to absorb and contain bodily waste in order to prevent soiling of the body and clothing of the wearer, as well as bedding or other objects with which the wearer comes into contact. As the usage of disposable absorbent articles has expanded, their complexity has increased with the incorporation of additional features serving to enhance their performance and appearance. The costs of the materials and the costs of the manufacturing processes have also increased in conjunction with the increase in complexity. As a result, the prices at which these articles are sold have risen to levels that many potential purchasers around the world cannot afford to pay. Thus, a need exists for a simple disposable absorbent article.

### SUMMARY OF THE INVENTION

A disposable absorbent article includes a chassis and an absorbent assembly and is adapted to be configured as a loincloth diaper for use in combination with a support element that is worn around the waist of the wearer. The chassis has fastening elements for use in forming laterally extending tunnels adjacent to the waist edges through which the support element extends. The chassis includes a water-impermeable backsheet that may be folded laterally inward at both of its side edges to form opposing side flaps. Each side flap is attached to the interior surface of the chassis adjacent to its end edges. Each side flap has a longitudinally extending elastic gathering member attached adjacent to its proximal edge. The chassis may include an extensible formed web material. The absorbent assembly may be attached in a cruciform pattern to the chassis to allow unattached portions of the chassis to extend laterally.

### BRIEF DESCRIPTION OF THE DRAWINGS

In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

FIG. 1 is a plan view of an exemplary disposable absorbent article in the form of a loincloth diaper 20, which is shown in its flat, uncontracted state, i.e., without the contraction induced by elastic members. In FIG. 1, the interior of the diaper is shown facing the viewer.

FIG. 2 is a plan view of the diaper 20 of FIG. 1 in its flat, uncontracted state, with the exterior portion of the diaper 20 shown facing the viewer.

FIG. 3 is a section view of the diaper 20 of FIG. 1 taken at the section line 3-3.

FIG. 4 is a section view of the diaper 20 of FIG. 1 taken at the section line 4-4.

FIG. 5 is a section view of the diaper 20 of FIG. 1 taken at the section line 5-5.

FIG. 6 is a section view of the diaper 20 of FIG. 1 taken at the section line 6-6.

FIG. 7 is a simplified side elevation view of an exemplary diaper 20 being worn about a lower torso of a wearer.

FIG. 8 is a front elevation view of the diaper 20 of FIG. 7.

FIG. 9 is a back elevation view of the diaper 20 of FIG. 7.

FIG. 10 is a perspective view of an exemplary diaper 20, which is shown in its relaxed, contracted state, i.e., with the contraction induced by elastic members. In FIG. 10, the interior portion of the diaper 20 is shown facing upward.

FIG. 11 is a plan view of an exemplary fragment of a formed web material.

FIG. 12 is a view of an exemplary diaper taken at the section line 12-12 of FIG. 8.

FIG. 13 is a view of another exemplary diaper taken at the section line 13-13 of FIG. 8.

FIG. 14 is a view of an exemplary diaper taken at the section line 14-14 of FIG. 9.

FIG. 15 is a view of another exemplary diaper taken at the section line 15-15 of FIG. 9.

FIG. 16 is a plan view of an exemplary diaper 20 shown in its flat, uncontracted state.

FIG. 17 is a section view of the diaper 20 of FIG. 16 taken at the section line 17-17.

FIG. 18 is a plan view of an exemplary diaper 20 shown in its flat, uncontracted state.

FIG. 19 is a section view of the diaper 20 of FIG. 18 taken at the section line 19-19.

FIG. 20 is a plan view of an exemplary diaper 20 shown in its flat, uncontracted state.

FIG. 21 is a plan view of an exemplary diaper 20 shown in its flat, uncontracted state.

FIG. 22 is a plan view of an exemplary diaper 20 shown in its flat, uncontracted state.

FIG. 23 is a plan view of an exemplary diaper 20 shown in its flat, uncontracted state.

FIG. 24 is a plan view of an exemplary diaper 20 shown in its flat, uncontracted state.

FIG. 25 is a plan view of an exemplary absorbent assembly 200 with the interior portion of the absorbent assembly 200 is shown facing the viewer.

FIG. 26 is a view of the absorbent assembly of FIG. 25 taken at the section line 26-26.

FIG. 27 is a view of the absorbent assembly of FIG. 25 taken at the section line 27-27.

FIG. 28 is a section view of an exemplary absorbent assembly 200.

FIG. 29 is a section view of an exemplary absorbent assembly 200.

### DETAILED DESCRIPTION OF THE INVENTION

In this description, the following terms have the following meanings:

The term “absorbent article” refers to a device that absorbs and contains liquid, and more specifically, refers to a device that is placed against or in proximity to the body of the wearer to absorb and contain the various exudates discharged from the body.

The term “diaper” refers to an absorbent article that is generally worn by infants and incontinent persons about the lower torso and that is specifically adapted to receive and contain urinary and fecal waste.

The term “disposable” refers to the nature of absorbent articles that generally are not intended to be laundered or otherwise restored or reused as an absorbent article, i.e., they are intended to be discarded after a single use and, preferably, to be recycled, composted or otherwise disposed of in an environmentally compatible manner.

The term “longitudinal” refers to a direction running from a waist edge to an opposing waist edge of the article and generally parallel to the maximum linear dimension of the article. Directions within ±45° of the longitudinal direction are considered to be “longitudinal”.

The term “lateral” refers to a direction running from a side edge to an opposing side edge of the article and generally at a right angle to the longitudinal direction. Directions within ±45° of the lateral direction are considered to be “lateral”.

The term “disposed” refers to an element being attached and positioned in a particular place or position in a unitary structure with other elements.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently.

The term “cohesive” refers to the property of a material that sticks to itself but does not to any significant degree stick to other materials.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables and Nonwovens Association).

The terms “proximal” and “distal” refer respectively to the location of an element near to or far from the center of a structure, e.g., the proximal edge of a longitudinally extending element is located nearer to the longitudinal axis than the distal edge of the same element is located relative to the same longitudinal axis.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower”, “above” and “below”, “over” and “under”, and “top” and “bottom”, respectively.

In the following description and in the drawing figures, various structural elements are identified by reference numerals without suffixed letters when referring to the group as a whole and by the same reference numerals with suffixed letters when distinguishing between, for example, left and right members of the group. As an example, the side flaps as a group are identified by the reference numeral 147 while the individual left and right side flaps are respectively designated as elements 147a and 147b.

### Description of Exemplary Loincloth Diaper Embodiment

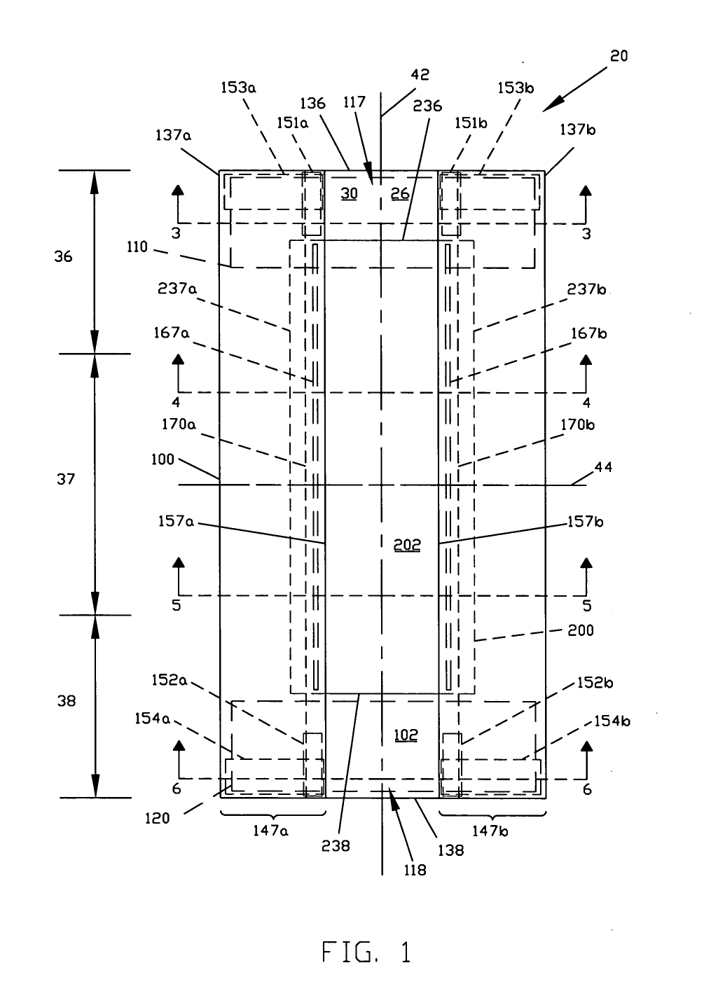

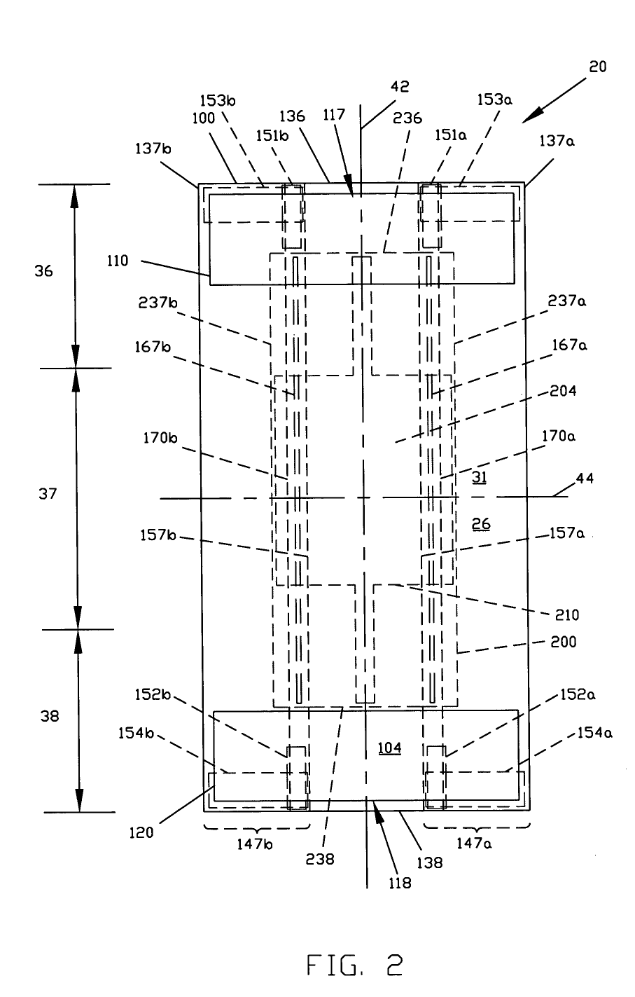

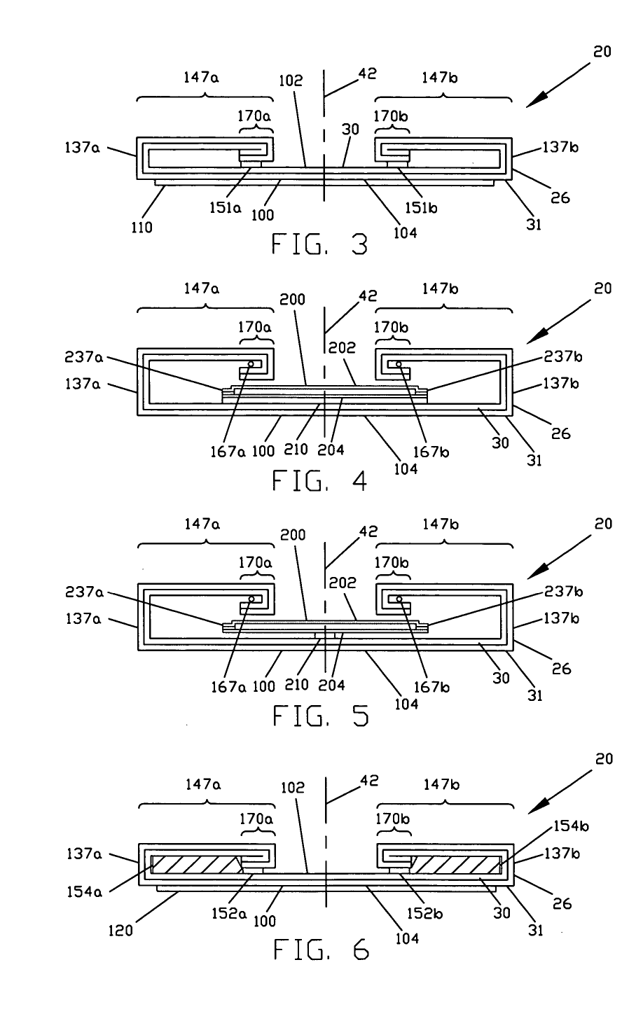

Reference is made to FIG. 1, FIG. 2, FIG. 3, FIG. 4, FIG. 5, and FIG. 6 for this section of this description.

One end portion of the exemplary loincloth diaper 20 is configured as a front waist region 36. The longitudinally opposing end portion is configured as a back waist region 38. An intermediate portion of the loincloth diaper 20 extending longitudinally between the front waist region 36 and the back waist region 38 is configured as a crotch region 37.

The basic structure of the loincloth diaper 20 includes a chassis 100. The chassis 100 has a laterally extending front waist edge 136 in the front waist region 36 and a longitudinally opposing and laterally extending back waist edge 138 in the back waist region 38. The chassis 100 has a longitudinally extending left side edge 137a and a laterally opposing and longitudinally extending right side edge 137b,both chassis side edges extending longitudinally between the front waist edge 136 and the back waist edge 138. The chassis 100 has an interior surface 102 and an exterior surface 104. The chassis 100 also has a longitudinal axis 42 and a lateral axis 44. The longitudinal axis 42 extends through the midpoint of the front waist edge 136 and through the midpoint of the back waist edge 138. The lateral axis 44 extends through the midpoint of the left side edge 137a and through the midpoint of the right side edge 137b. The exemplary chassis 100 shown in FIG. 1 additionally has longitudinally extending and laterally opposing side flaps 147a and 147b that are described in more detail below.

The basic structure of the loincloth diaper 20 also includes an absorbent assembly 200 that is attached to the chassis 100. The absorbent assembly 200 has a laterally extending front edge 236 in the front waist region 36 and a longitudinally opposing and laterally extending back edge 238 in the back waist region 38. The absorbent assembly 200 has a longitudinally extending left side edge 237a and a laterally opposing and longitudinally extending right side edge 237b,both absorbent assembly side edges extending longitudinally between the front edge 236 and the back edge 238. The absorbent assembly 200 has an interior surface 202 and an exterior surface 204. The absorbent assembly 200 may be disposed symmetrically with respect to either or both of the longitudinal axis 42 and the lateral axis 44. Alternatively, the absorbent assembly 200 may be disposed asymmetrically with respect to either or both of the longitudinal axis 42 and the lateral axis 44. For example, the absorbent assembly 200 shown in FIG. 1 is disposed symmetrically with respect to the longitudinal axis 42 and asymmetrically with respect to the lateral axis 44. In particular, the absorbent assembly 200 shown in FIG. 1 is disposed asymmetrically toward the front waist region 36.

The respective front edge 236, back edge 238, left side edge 237a,and right side edge 237b of the absorbent assembly 200 may lie inward of the respective front waist edge 136, back waist edge 138, left side edge 137a,and right side edge 137b of the chassis 100, as in the exemplary loincloth diaper 20 shown in FIG. 1. Such a configuration in which one or more of the edges of the absorbent assembly 200 lies inward of the corresponding edges of the chassis 100 may be desirable, for example, in order to allow the relatively more flexible layer or layers adjacent to the edges of the chassis to conform to the body of the wearer and thereby form effective gasket-like seals against the skin of the wearer without being constrained by a relatively thicker and relatively less flexible absorbent assembly. Alternatively, one or more of the edges of the absorbent assembly 200 may coincide with the corresponding edge or edges of the chassis 100.

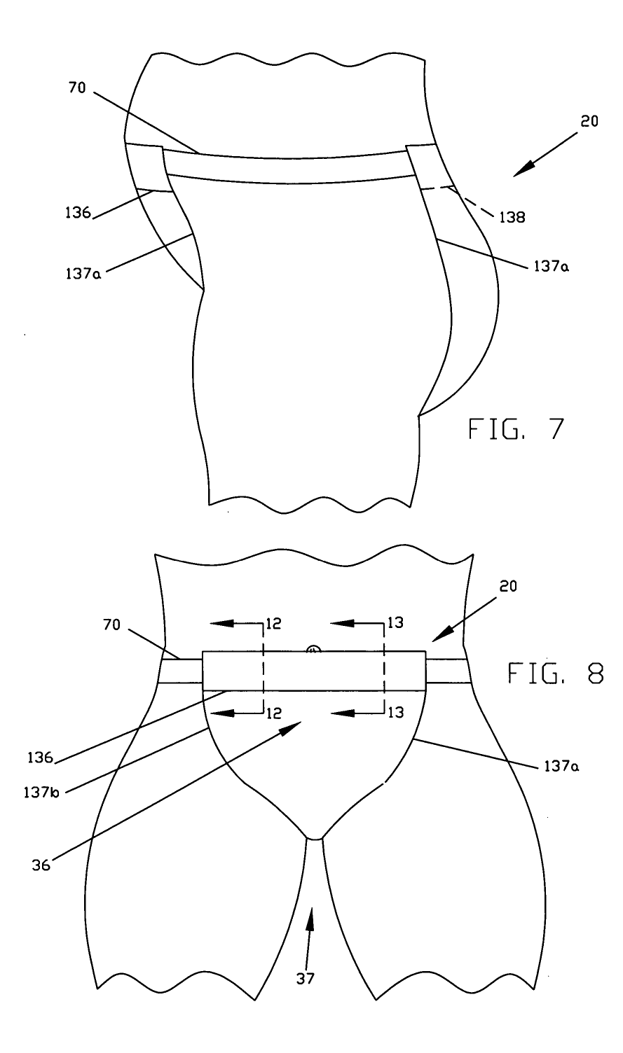

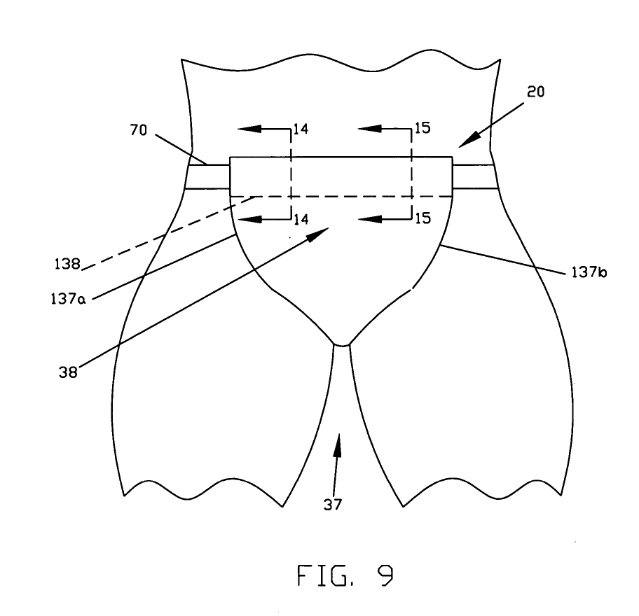

As shown in FIG. 7, FIG. 8, and FIG. 9, when the loincloth diaper 20 is worn on the lower torso of a wearer, the front waist edge 136 and the back waist edge 138 partially encircle the waist of the wearer, while at the same time the chassis side edges 137a and 137b partially encircle the legs of the wearer. At the same time, the crotch region 37 is generally positioned between the legs of the wearer and the absorbent assembly 200 extends from the front waist region 36 through the crotch region 37 to the back waist region 38.

Description of the Chassis

The chassis 100 includes a water-impermeable backsheet 26. The backsheet 26 forms an exterior surface that is intended to be placed toward any clothing that is worn over the loincloth diaper 20. Many suitable materials for use as the backsheet 26 are well-known, including films of polyethylene and other polyolefins. Multi-layer backsheets, such as a laminate of a film 30 and a nonwoven material 31 or a laminate of multiple nonwoven layers, may also be suitable for use as the backsheet 26. Such a backsheets may be oriented with the nonwoven 31 disposed exteriorly of the film, as shown in FIG. 1, FIG. 2, FIG. 3, FIG. 4, FIG. 5, FIG. 6, and FIG. 14, to provide the feel and appearance of a more cloth-like outermost layer than would be provided by using the film 30 as the outermost layer.

The chassis 100 may, but need not, additionally include an inner liner 22 attached to the backsheet 26. The inner liner 22 may form a portion of the interior surface 102 of the chassis 100 that is intended to be placed against the body of the wearer. For example, the inner liner may cover and thereby lie interiorly of a portion or all of the absorbent assembly 200. The inner liner 22 preferably is formed of a soft material that will not irritate the skin of the wearer. Such an inner liner 22 may serve to isolate the skin of the wearer from a portion of the backsheet 26 as may be desirable, for example, when the loincloth diaper 20 is worn under conditions in which contact between the skin and a backsheet film could be uncomfortable. Many suitable materials for the inner liner 22 are well-known in the art, including rayon and synthetic nonwovens such as spunbonded or carded polypropylene or polyester or other olefinic materials. The inner liner 22 may extend to the same width and the same length as the backsheet 26. Alternatively, one or more of the edges of the inner liner 22 may lie inward of the edges of the backsheet 26.

As shown in FIG. 1, FIG. 2, FIG. 3, FIG. 4, FIG. 5, and FIG. 6, the exemplary chassis 100 has longitudinally extending and laterally opposing side flaps 147a and 147b that are disposed on the interior portion of the loincloth diaper 20 that faces inwardly toward the wearer and contacts the wearer. The side flaps 147 may be formed by folding portions of the chassis 100 laterally inward, i.e., toward the longitudinal axis 42, to form both the respective side flaps 147 and the side edges 137 of the chassis 100. Alternatively, the side flaps 147 may be formed by attaching an additional layer or layers to the chassis 100 at or adjacent to each of the respective side edges 137 of the chassis 100. In embodiments in which the side flaps are formed by attaching an additional layer or layers to the chassis, each of the additional layer or layers may be attached at or adjacent to its laterally distal edge.

Portions of a film backsheet 26 that are folded laterally inward to form the side flaps may contact the skin of a wearer during the use of the loincloth diaper 20. However, in embodiments comprising an extensible backsheet, the alternating ridges and valleys in such a film backsheet that has been deformed in order to make it extensible may provide channels through which air can pass to alleviate any concern regarding such contact of the film backsheet with the skin.

In embodiments in which portions of the chassis 100 are folded laterally inward to form the side flaps 147, the chassis 100 may simply be folded loosely or may be creased along a portion of each of its side edges 137. For example, it may be desirable to form creases along portions of the side edges 137 in the crotch region 37 in order to impart a more finished appearance to the loincloth diaper 20. Alternatively or in addition to creasing, a portion of each of the folded side flaps 147 adjacent to the side edges 137 may be attached to the interior surface 102 of the chassis 100 to achieve a similar result.

The left side flap 147a has a proximal edge 157a and the right side flap 147b has a proximal edge 157b. In the exemplary loincloth diaper 20 shown in FIG. 1, the side flaps 147 overlap the absorbent assembly 200, i.e., the proximal edges 157 lie laterally inward of the respective side edges 237 of the absorbent assembly 200. Such an overlapped configuration may be desirable in order to impart a more finished appearance to the loincloth diaper 20 than that imparted by a non-overlapped configuration. Alternatively, the side flaps 147 may not overlap the absorbent assembly 200.

In the exemplary chassis 100 shown in FIG. 1, the side flaps 147 extend the full length of the chassis 100 between the front waist edge 136 and the back waist edge 138. Such a full length configuration may be desirable in order to minimize the amount of waste material and the difficulty associated with the manufacture of the loincloth diaper 20, especially when the method used to manufacture the loincloth diaper 20 requires the introduction of the material or materials for the chassis 100 in the form of a continuous web or multiple continuous webs. Alternatively, the side flaps may be shorter and extend less than the full distance between the front waist edge 136 and the back waist edge 138. Such a shorter configuration may be desirable in order to minimize the total amount of material used in the manufacture of the loincloth diaper 20.

Each of the side flaps 147 is attached to the interior surface 102 of the chassis 100 in attachment zones located in the front waist region 36 and in the back waist region 38. For example, in the chassis 100 shown in FIG. 1, the side flaps 147 are attached to the interior surface 102 of the chassis 100 in the attachment zones 151. These longitudinally oriented attachment zones may have equal areas or may be unequal in area. For example, the front longitudinally oriented attachment zones 151 may be of one size and the back longitudinally oriented attachment zones 152 may be of another size.

In the exemplary chassis 100 shown in FIG. 1, the side flaps 147 are also attached to the interior surface 102 of the chassis 100 in laterally oriented attachment zones 153 adjacent to the front waist edge 136 and in a longitudinally opposing laterally oriented attachment zones 154 adjacent to the back waist edge 138. These laterally oriented attachment zones may have equal areas or may be unequal in area.

Alternatively, each attachment zone may extend laterally across the full width of the respective side flap. For example, a laterally oriented attachment zone may extend laterally from the chassis left side edge 137a to the left side flap edge 157a and thereby attach the entire width of the left side flap 147a adjacent to the front waist edge 136 to the interior surface 102 of the chassis 100. In embodiments in which the front edge 236 or the back edge 238 of the absorbent assembly 200 coincides with the respective front waist edge 136 or back waist edge 138 of the chassis 100 and the side flaps 147 overlap the absorbent assembly 200, the side flaps 147 may be attached to the absorbent assembly 200 instead of, or in addition to, being attached to the interior surface 102 of the chassis 100.

Between the attachment zones, the proximal edges 157 of the side flaps 147 remain free, i.e., are not attached to the interior surface 102 of the chassis 100 or to the absorbent assembly 200. Also between the attachment zones, each side flap preferably includes a longitudinally extensible flap elastic gathering member that is attached adjacent to the proximal edge of the side flap by any of many well-known means. Each such flap elastic gathering member may be attached over its entire length or over only a portion of its length. For example, such a flap elastic gathering member may be attached only at or near its longitudinally opposing ends and may be unattached at the middle of its length. Such a flap elastic gathering member may be disposed in the crotch region 37 and may extend into one or both of the front waist region 36 and the back waist region 38. For example, in the exemplary chassis 100 shown in FIG. 1, an elastic strand 167 is attached adjacent to the proximal edge 157 of each of the side flaps 147 and extends into both the front waist region 36 and the back waist region 38.

Each flap elastic gathering member may be enclosed inside a folded hem. For example, in the exemplary chassis 100 shown in FIG. 4 and FIG. 5, each of the elastic strands 167 is enclosed inside a hem 170 formed adjacent to the proximal edge 157 of the respective side flap 147. Alternatively, the flap elastic gathering member may be sandwiched between two layers of the chassis, e.g., between the layers of a laminate backsheet or between a backsheet and an inner liner. As another alternative, the flap elastic gathering member may be attached on a surface of the chassis 100 and remain exposed.

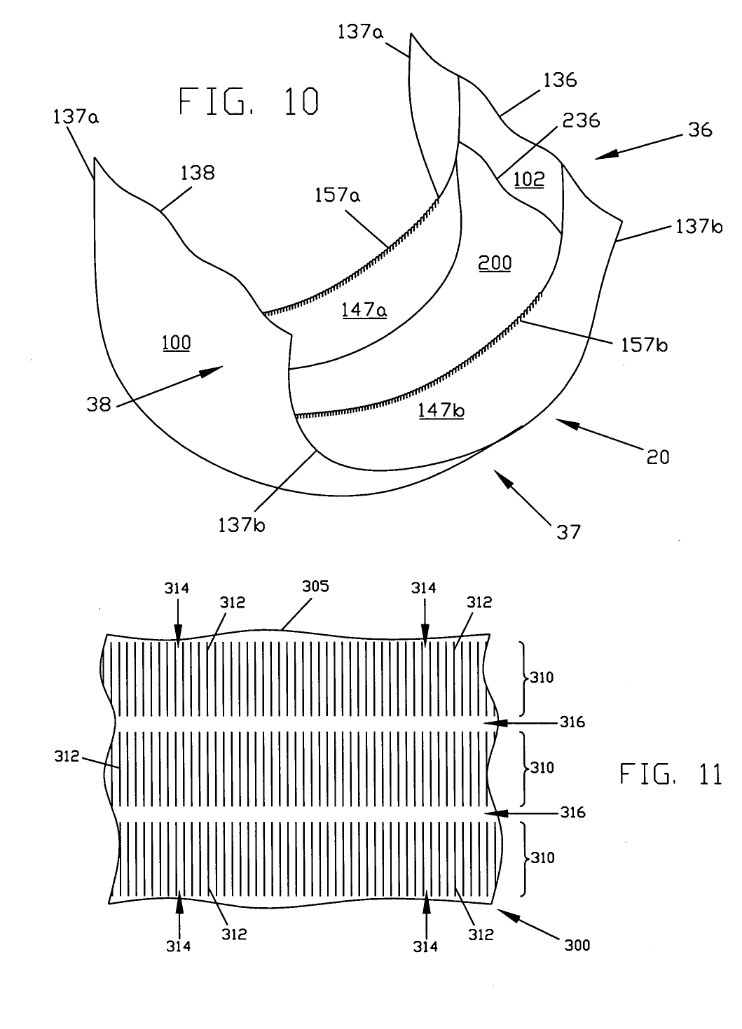

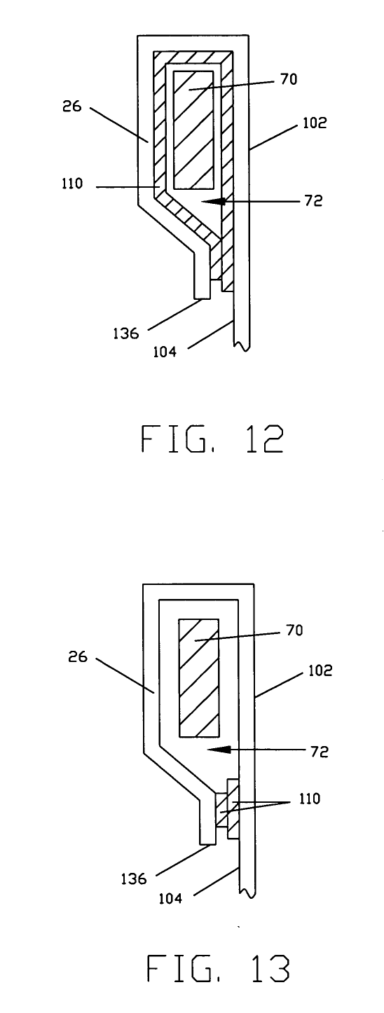

When stretched, the flap elastic gathering member adjacent to each side flap edge allows the side flap edge to extend to the flat uncontracted length of the chassis, e.g., the length of the chassis 100, as shown in FIG. 1. When allowed to relax, the flap elastic gathering member contracts to gather the portion of the side flap edge along which the flap elastic gathering member is attached and thereby make the relaxed length of the side flap edge less than the flat uncontracted length of the chassis. For example, when the exemplary loincloth diaper 20 is in a relaxed condition as shown in FIG. 10, the elastic strands 167 contract to gather the proximal edges 157 of the side flaps 147. The contractive forces of the elastic strands 167 are transmitted at the respective front attachment zones 151 and at the respective back attachment zones 152 to the interior surface 102 of the chassis 100. These contractive forces pull the front waist region 36 and the back waist region 38 toward each other and thereby bend the loincloth diaper 20 into a “U” shape in which the interior of the “U” shape is formed by the portions of the loincloth diaper 20 that are intended to be placed toward the body of the wearer. Because each of the proximal edges 157 remains free between the attachment zones 151 and 152, the contractive force of the elastic strand 167 lifts the proximal edge 157 away from the interior surface 102 of the chassis 100. As shown in FIG. 10, this lifting of the proximal edges 157 when the loincloth diaper 20 is in the relaxed condition lifts the side flaps 147 into position to serve as side barriers adjacent to the side edges 237 of the absorbent assembly 200.

When the loincloth diaper 20 is worn, the relaxed “U” shape generally conforms to the body of the wearer such that the front waist region 36 and the back waist region 38 are positioned such that they partially encircle the waist and the legs of the wearer. When the loincloth diaper 20 is worn in this manner, the elastic strands 167 tend to hold the lifted proximal edges 157 of the side flaps 147 in contact with the body of the wearer and thereby form seals to help prevent the leakage of deposited bodily waste out of the loincloth diaper 20. The lateral spacing of the lifted proximal edges 157 is selected to allow the deposit of bodily wastes from the lower torso of the wearer into the space between the lifted side flaps 147 and thereby directly onto the absorbent assembly 200. The width of each of the side flaps 147 in effect becomes its height when the free portion of its proximal edge is lifted and the side flap serves as a side barrier to leakage. This height preferably is selected to allow the lifted proximal edges 157 to fit into the leg creases of the body of the wearer at the same time as the absorbent assembly 200 is held in contact with the body.

In the finished loincloth diaper, the chassis may have a generally rectangular shape, as in the exemplary loincloth diaper 20 shown in FIG. 1 and FIG. 2. Such a generally rectangular configuration may be desirable in order to minimize the amount of waste material and the difficulty associated with the manufacture of the loincloth diaper 20.

A portion or the whole of the chassis 100 may be made extensible to a degree greater than the inherent extensibility of the material or materials from which the chassis is made, e.g, the backsheet 26, the inner liner 22, or both. The additional extensibility may be desirable in order to allow the chassis 100 to conform to the body of a wearer during movement by the wearer. The additional extensibility may also be desirable, for example, in order to allow the user of a loincloth diaper 20 including a chassis 100 having a particular size before extension to extend the front waist region 36, the back waist region 38, or both waist regions of the chassis 100 to provide additional body coverage for wearers of differing size, i.e., to tailor the loincloth diaper to the individual wearer. Such extension of the waist region or regions may give the loincloth diaper a generally hourglass shape, so long as the crotch region 37 is extended to a relatively lesser degree than the waist region or regions, and may impart a tailored appearance to the loincloth diaper 20 when it is worn. In addition, the additional extensibility may be desirable in order to minimize the cost of the loincloth diaper. For example, an amount of material that would otherwise be sufficient only to make a relatively smaller loincloth diaper lacking this extensibility can be used to make a loincloth diaper capable of being extended to adequately cover a wearer that is larger than the unextended, smaller, loincloth diaper would fit. In other words, a lesser amount of material is needed in order to make a loincloth diaper capable of providing adequate coverage for a given size of a wearer when the material is made extensible as described. The portion of the chassis in one of the waist regions may be made laterally extensible to a maximum extensibility greater than a maximum extensibility of another portion of the chassis in the crotch region such that a lateral extension of each of the portions to its maximum extensibility imparts an hourglass shape to the chassis.

Additional lateral extensibility in the chassis 100 may be provided in a variety of ways. For example, a material or materials from which the chassis 100 is made may be pleated by any of many known methods. Alternatively, all or a portion of the chassis may be made of a formed web material or a formed laminate of web materials like those described in U.S. Pat. No. 5,518,801 issued on 21 May 1996 in the name of Chappell et al. An exemplary fragment 300 of such a formed web material 305 is shown in FIG. 11. This formed web material 305 includes distinct laterally extending regions 310 in which the original material has been altered by embossing or another method of deformation to create a pattern of generally longitudinally oriented alternating ridges 312 and valleys 314. The formed web material 305 also includes laterally extending unaltered regions 316 located between the laterally extending altered regions 310.

The front laterally central portion 117 and the back laterally central portion 118 of the chassis 100 between the attachment zones 151, 152, 153, and 154 where the side flaps 147a and 147b are attached to the interior surface 102 of the chassis adjacent to the respective waist edges 137 and 138 may have a different range of extensibility from the portions of the chassis in the attachment zones. Additionally or alternatively, the laterally central portions 117 and 118 may be extensible to a greater or lesser degree when subjected to a given level of opposing tensile forces, i.e., may be more easily or less easily extensible, than the portions of the chassis in the attachment zones. For example, if the chassis is made uniformly extensible across its entire width prior to the formation of the side flaps, the double layering in the areas of the attachment zones after the formation of the side flaps may have an effect of decreasing the degree of lateral extensibility of those areas under a given level of opposing tensile forces, such as by the side flaps acting as parallel “springs” that must be extended in order to extend the underlying attached portion of the chassis. As another example, the altered regions in the laterally central portions of the chassis may be deformed to a greater or a lesser degree than the altered regions in the attachment zones to render the laterally central portions more easily or less easily extensible than the respective portions in the attachment zones.

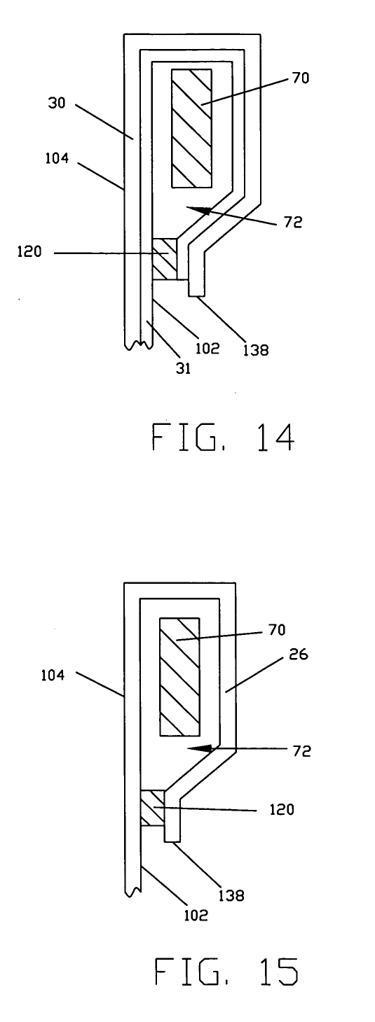

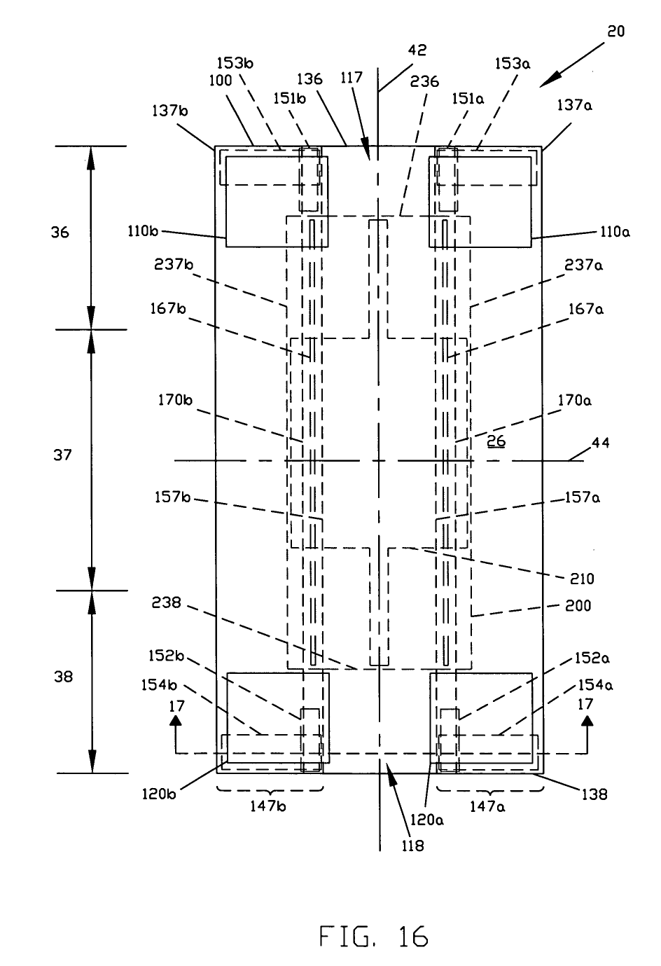

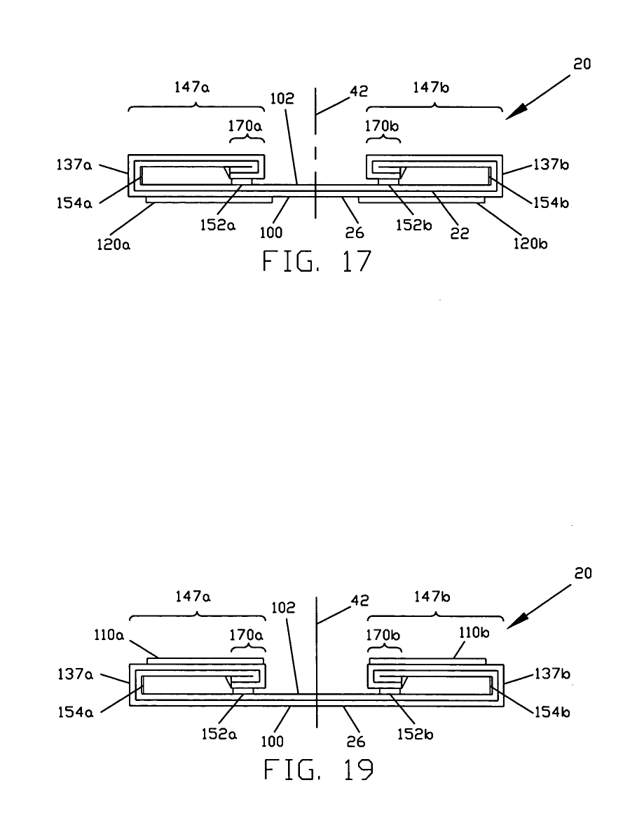

The chassis 100 has fastening elements in the front waist region 36 and the back waist region 38 for use in positioning the absorbent assembly 200 appropriately to receive and contain urinary and fecal waste excreted by the wearer. These fastening elements are used in combination with a separate support element that is worn around the waist of the wearer such that the disposable absorbent article 20 is configured like a loincloth. Therefore, the fastening elements are disposed adjacent to the front waist edge 136 and the back waist edge 138 of the chassis 100. As shown in FIG. 7, FIG. 8, and FIG. 9, each waist edge 136 and 138 is folded over and the fastening elements are engaged to maintain this folded-over configuration. Thus, the folding and the engagement of the fastening elements form laterally extending tunnels 72 through which the support element 70 extends, as shown in FIG. 12, FIG. 13, FIG. 14, and FIG. 15. The fastening elements preferably are openable and refastenable in order to facilitate the adjustment of the fit of the loincloth diaper onto the body of the wearer and to facilitate the eventual removal of the loincloth diaper from the wearer for disposal.

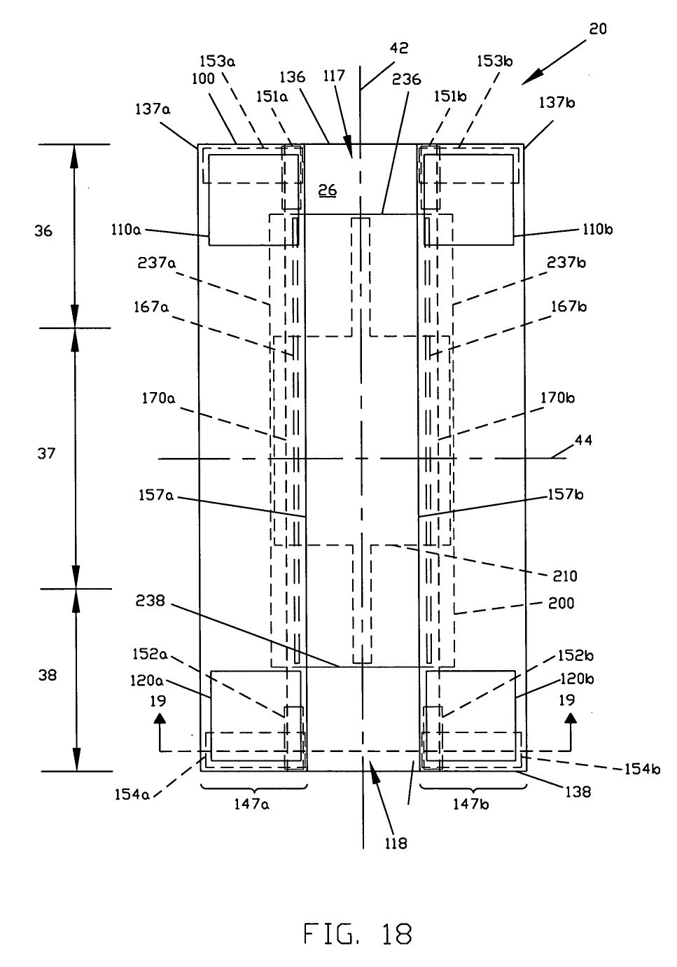

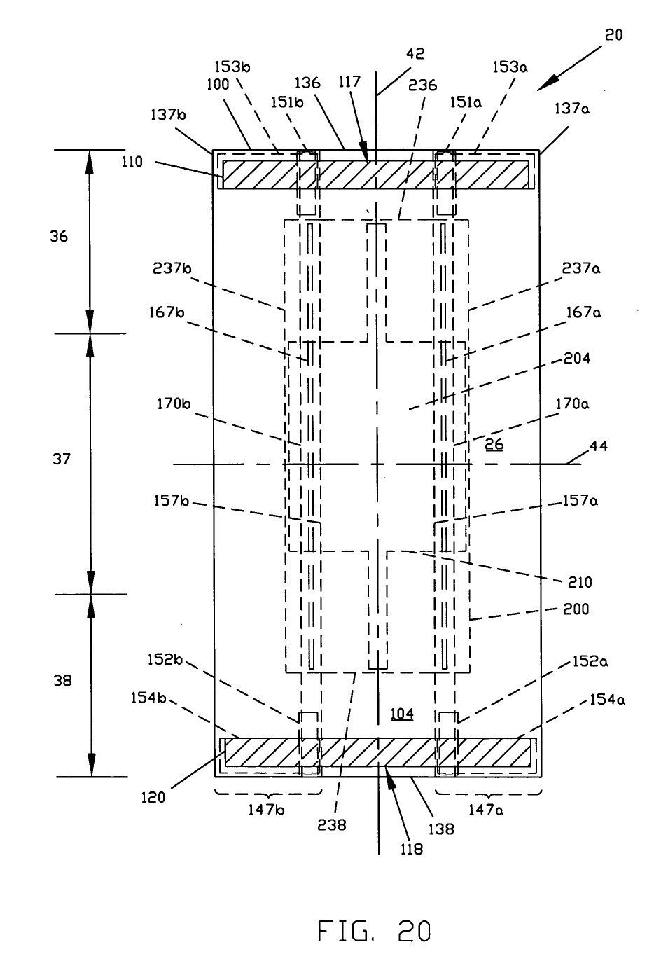

As shown in FIG. 1, FIG. 2, FIG. 3, FIG. 6, FIG. 12, FIG. 13, FIG. 14, FIG. 15, FIG. 16, FIG. 17, FIG. 18, and FIG. 19, the chassis 100 has longitudinally opposing fastening elements 110 and 120 respectively disposed adjacent to its front waist edge 136 and its back waist edge 138. Such fastening elements may be laterally continuous, i.e., have the form of a single fastening element adjacent to each waist edge, as shown in FIG. 1, FIG. 2, FIG. 3, and FIG. 6. Alternatively, each fastening element may be laterally discontinuous, i.e., have the form of two or more laterally discrete segments. For example, in FIG. 16, FIG. 17, FIG. 18, and FIG. 19, each fastening element 110 and 120 includes two laterally discrete segments. The use of a single laterally continuous fastening element adjacent to each waist edge may provide a relatively more finished appearance when the loincloth diaper is worn. On the other hand, when ends of the support element must be tied together for use, the use of laterally discretely segmented fastening elements adjacent to each waist edge may allow relatively more flexibility for locating the knot(s), e.g., the knot(s) can be located between the laterally discrete segments, if desired. Of course, any knot(s) can likewise be located laterally distally relative to the side edges 137 of the chassis.

The fastening elements 110 and 120 may be disposed on the exterior surface 104 of the chassis 100 as shown in FIG. 1, FIG. 2, FIG. 3, FIG. 6, FIG. 12, FIG. 13, FIG. 16, and FIG. 17, as well as for the front fastening element(s) 110 in FIG. 7 and FIG. 8. In such an embodiment, as shown in FIG. 12 and FIG. 13, the waist edges 136 and 138 are folded over from inside-to-outside to engage the fastening elements 110 and 120 and thereby form the laterally extending tunnels 72, such that the exterior surface 104 lies inside the tunnels 72. Alternatively the fastening elements may be disposed on the interior surface 102 of the chassis 100 as shown in FIG. 14, FIG. 15, FIG. 18, and FIG. 19, as well as for the back fastening element(s) 120 in FIG. 8 and FIG. 9. In such an embodiment, as shown in FIG. 14 and FIG. 15, the waist edges 136 and 138 are folded over from outside-to-inside to engage the fastening elements 110 and 120 and thereby form the laterally extending tunnels 72, such that the interior surface 102 lies inside the tunnels 72. Alternatively, one of the waist edges may be folded inside-to-outside and the opposing waist edge may be folded outside-to-inside.

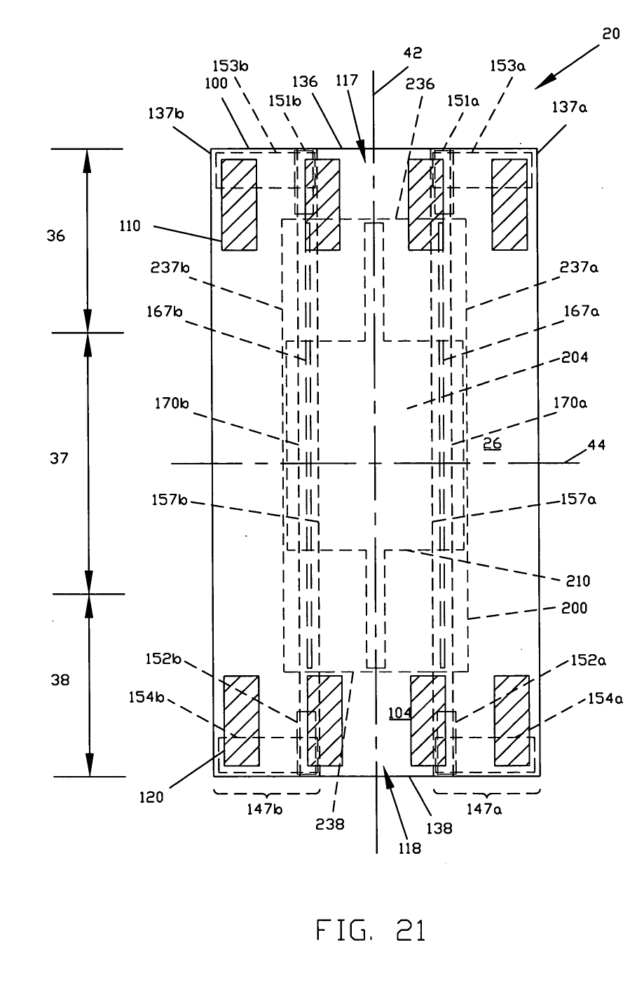

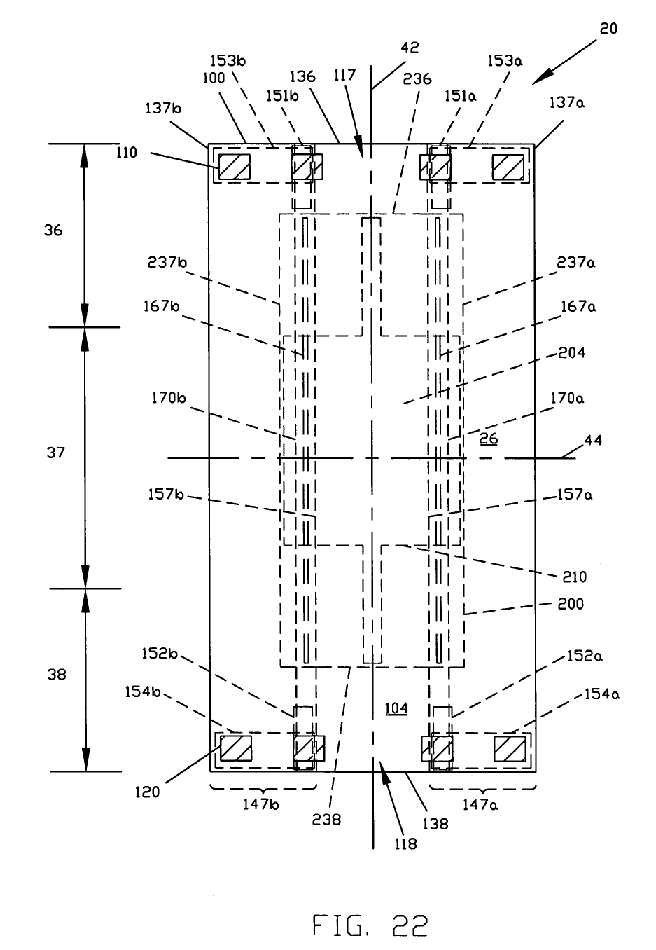

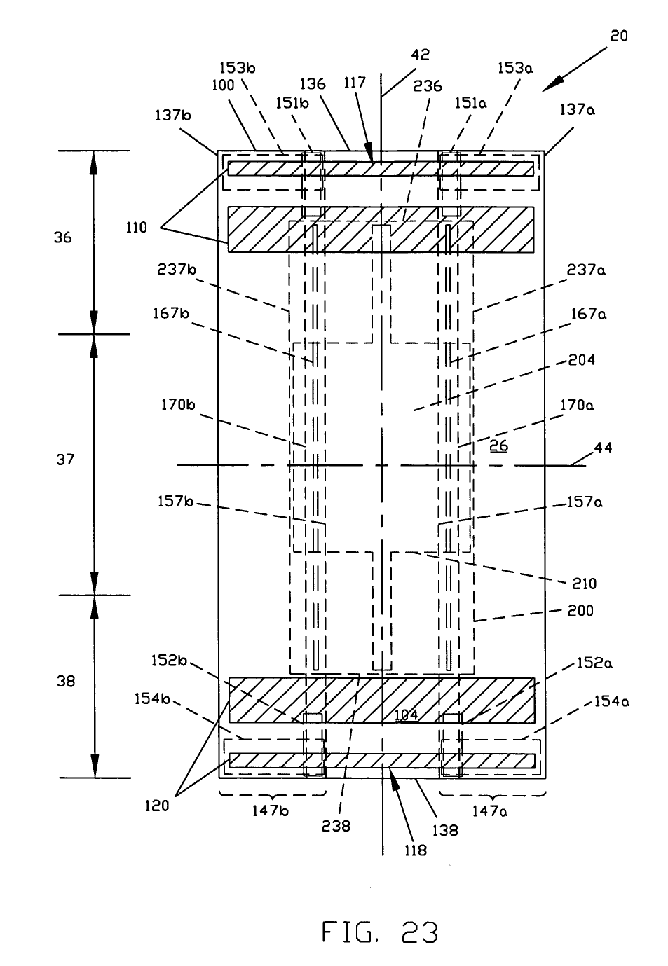

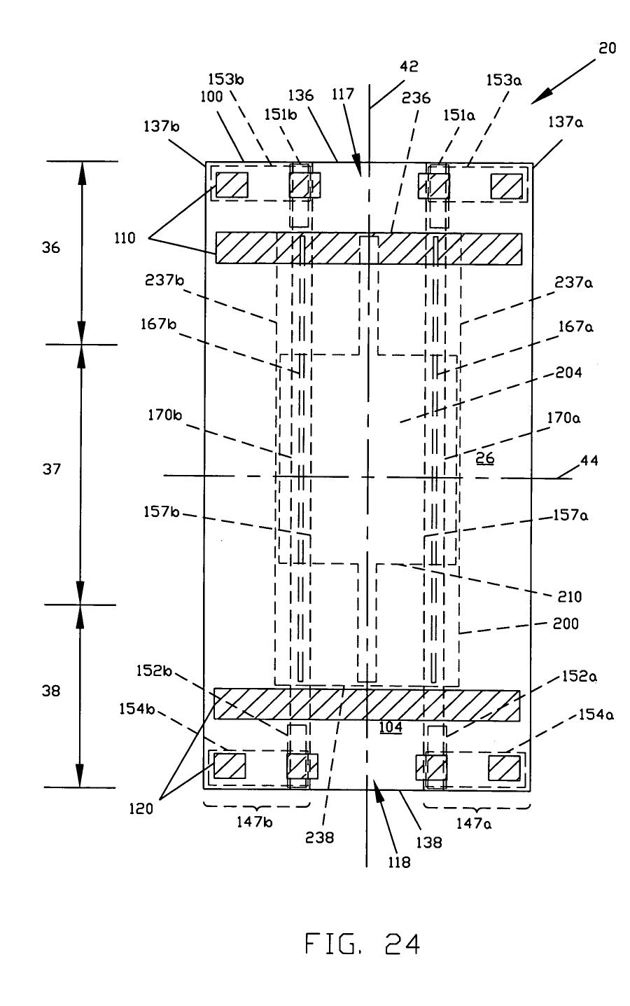

The fastening elements 110 and 120 may be disposed in various alternative configurations, in addition to those previously described. Several additional exemplary configurations are shown in FIG. 20, FIG. 21, FIG. 22, FIG. 23, and FIG. 24, all of which show views of the exterior surface 104 of the chassis 100. The fastening elements may likewise be disposed on the interior surface 102 of the chassis 100 in any of these exemplary configurations. In FIG. 20, a single laterally continuous fastening element is shown adjacent to each waist edge, with each of these fastening elements extending longitudinally less far than the previously described fastening elements. Such “shorter” fastening elements may allow the waist edges to be folded over more easily by providing less bending resistance. In FIG. 21, laterally discretely segmented fastening elements are shown. Such a configuration may allow relatively easy adjustability. In FIG. 22, “shorter” laterally discretely segmented fastening elements are shown. In FIG. 23, longitudinally discontinuous fastening elements are shown, such that a “shorter” laterally continuous fastening element is disposed longitudinally outwardly of a relatively “longer” laterally continuous fastening element in each waist region. The “shorter” fastening element may be attached to the “longer” fastening element in a variety of positions and thus this configuration may provide adjustability for use with support elements of different widths. In FIG. 24, a combination of “shorter” laterally discretely segmented fastening elements disposed adjacent to the waist edges and “shorter” laterally continuous fastening elements disposed relatively longitudinally proximally is shown, as one example of the many possible combinations.

The fastening elements 110 and 120 can be incorporated into the chassis 100 to enable a user to apply the loincloth diaper 20 to the body of the wearer without, or in conjunction with, any separate fastening devices. Many suitable types of such incorporated fastening elements are well-known, including, for example, patches of adhesive materials, buttons, hooks, loops, snap fasteners, other forms of mechanical fasteners, patches of cohesive materials, patches of relatively low tack peelable hot melt adhesive materials, etc. In general, any type of fastening element used in conventional disposable diapers to fasten the front waist region 36 and the back waist region 38 together to encircle the waist and the legs of the wearer can be used in the present invention to form the tunnels 72.

For example, an exemplary one-piece fastening element 110 that attaches to itself is shown in FIG. 12, an exemplary two-piece fastening element 110 in which the two pieces attach to each other is shown in FIG. 13, an exemplary one-piece fastening element 120 that attaches to a nonwoven layer 31 of a laminate backsheet is shown in FIG. 14, and a one-piece fastening element 120 that attaches to any form of a backsheet 26 is shown in FIG. 15. When the exterior surface 104 of the chassis 100 is formed by a nonwoven material 31 as in the embodiment shown in FIG. 14, the fastening elements 110 and/or 120 may include hooks adapted to engage with the nonwoven material, thereby making it unnecessary to use loops specifically placed to engage with the hooks.

Exemplary fastening elements in the form of cohesive fastening patches may be formed of an inherently crystalline water-based synthetic elastomer to which a tackifying agent has been added to disrupt the polycrystalline structure and thereby render the elastomer cohesive. Such synthetic cohesive products are available from Andover Coated Products, Incorporated, of Salisbury, Mass., U.S.A. and are described in U.S. Pat. No. 6,156,424 issued on 5 Dec. 2000 in the name of Taylor. The attachment means may comprise attachment patches formed by the application of a cohesive material directly onto the chassis or onto a separate substrate that is in turn attached to the chassis. For example, a “hot melt” cohesive material may be applied in its molten state onto a surface and allowed to cool and solidify to form such a patch. The cohesive material may be applied in any of a variety of patterns, such as a continuous film, discrete dots, stripes, polygons, etc., and/or spaced and interconnected geometric elements describing a grid.

Description of the Support Element

The support element 70 is intended to encircle the waist of the wearer such that when extended through the laterally extending tunnels 72 formed by folding over the waist edges 136 and 138 and engaging the fastening elements 110 and 120, the support element can be used to support the waist regions 36 and 38 of the loincloth diaper 20 adjacent to the waist of the wearer and thereby position the absorbent assembly 200 appropriately to receive and contain urinary and fecal waste excreted by the wearer.

The support element can take the form of a belt, a cord, a sash, a band, a strap, an elasticated version of any of these forms, a combination of any of these forms, or any other form of waist-encircling element suitable for holding the disposable absorbent article 20 against the body of the wearer in the well-known orientation of a loincloth. For example, the support element may have the form of a closed loop, such as an elastic band, or alternatively the support element may be discontinuous in form and configured as a filament or a string having two ends that can be attached together to form a closed loop about the waist of the wearer. Additionally, the support element can have the form of two or more pieces that are attached together to form a closed loop. The support element is preferably formed of an elastic material that is either continuous, forming a closed loop about the waist, or alternatively the elastic can be tied about the waist of the wearer.

The support element may be packaged with the loincloth diaper for sale, e.g., a single support element or several support elements may be provided in a package containing several loincloth diapers. The loincloth diaper may be pre-assembled with the support element extending through the tunnel, or the support element may be separately provided by the caregiver. The support element may be formed of a non-durable material or materials and thereby be intended to be discarded after a single use or, alternatively, the support element may be made of durable materials that can be reused, including after laundering.

Description of the Absorbent Assembly

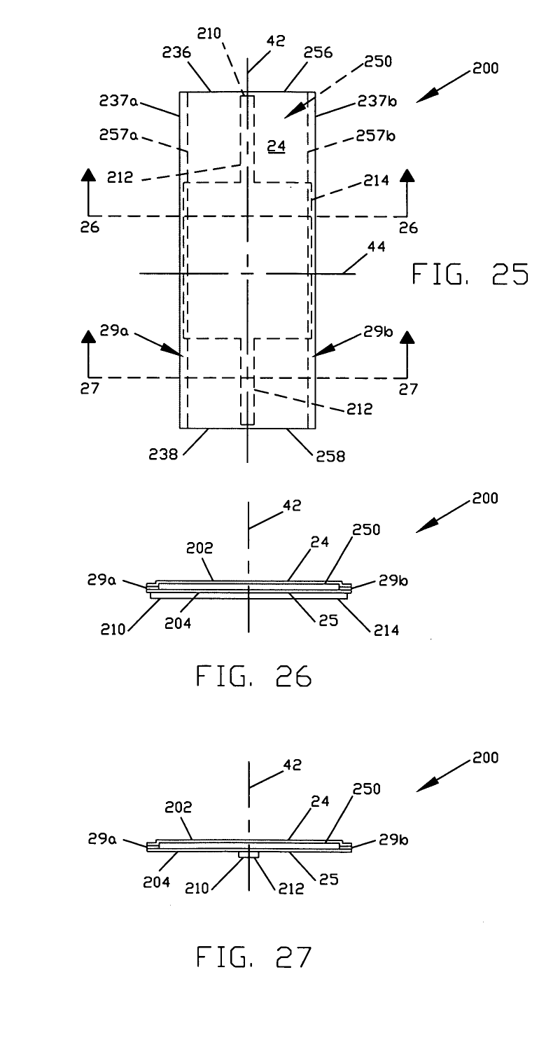

As shown in FIG. 25, FIG. 26, and FIG. 27, the absorbent assembly 200 includes an absorbent core 250 that serves to absorb and retain liquid bodily waste materials. The absorbent core 250 has a laterally extending front edge 256 and a longitudinally opposing and laterally extending back edge 258. The absorbent core 250 also has a longitudinally extending left side edge 257a and a laterally opposing and longitudinally extending right side edge 257b,both absorbent core side edges extending longitudinally between the front edge 256 and the back edge 258. The absorbent core 250 also has an interior surface 252 and an exterior surface 254.

The absorbent assembly 200 may be attached to the interior surface 102 of the chassis 100 over any part or the whole of the area of the absorbent assembly 200. Preferably, the absorbent assembly 200 is attached on its exterior surface 204 to the chassis 100 in a cruciform attachment pattern, i.e., in an attachment pattern that forms or is arranged in a cross or “+” shape. The cruciform attachment pattern may be contiguous, i.e., all of its portions may be touching or connected throughout the pattern in an unbroken sequence. Alternatively, the cruciform attachment pattern may include detached portions and thereby lack contiguity but still be arranged such that the shape of the overall pattern is a cruciform. For example, a discontiguous cruciform attachment pattern may include a longitudinally extending portion disposed along the longitudinal axis and separate left and right laterally distal portions disposed along or adjacent to the lateral axis and thereby form a cruciform as the shape of the overall pattern.

An exemplary contiguous cruciform attachment pattern 210 is shown in FIG. 2, FIG. 4, FIG. 5, FIG. 16, FIG. 18, FIG. 20, FIG. 21, FIG. 22, FIG. 23, FIG. 24, FIG. 25, FIG. 26, and FIG. 27. The portions of the chassis 100 that lie outside such a cruciform attachment pattern are not restrained by attachment to the absorbent assembly 200 and therefore remain extensible. In particular, a relatively narrow longitudinally extending portion 212 of a cruciform attachment pattern 210 like that shown in these figures leaves the majority of the width of the chassis 100 in the front waist region 36 and in the back waist region 38 freely extensible and thereby allows extension of the chassis 100 in the lateral direction in these regions. A relatively wide laterally extending portion 214 of a cruciform attachment pattern 210 like that shown in these figures prevents the portion of the chassis 100 in the crotch region 37 to which the absorbent assembly 200 is attached from shifting relative to the absorbent assembly 200 in that region. A relatively wide laterally extending portion 214 of a cruciform attachment pattern 210 may also contribute to the effectiveness and positioning of the side flaps 147 when the elastic strands 167 lift the proximal edges 157 into contact with the body of the wearer. For example, if the absorbent assembly was attached only along the longitudinal centerline, the absorbent assembly could be compressed by the legs to a smaller lateral dimension than desired. This narrowing of the absorbent assembly would in turn allow the chassis 100 in the crotch region 37 to narrow, i.e., allow the side edges 137 to move toward the longitudinal axis 42. Such narrowing of the chassis 100 would increase the likelihood that the side flaps 147 would distort and fail to maintain contact with the body and/or become improperly positioned. However, because the relatively wide laterally extending portion 214 of the cruciform attachment pattern 210 restrains the chassis 100 over a relatively wide portion of the width of the crotch region 37, the side flaps 147 are more likely to remain properly positioned while being lifted by the elastic strands 167.

Within the extent of the cruciform attachment pattern 210, the absorbent assembly 200 may be attached to the chassis 100 continuously or intermittently. For example, a film of an adhesive material may be applied continuously over the entire area of the cruciform attachment pattern and then used to continuously attach the absorbent assembly to the chassis. As an alternative example, an adhesive material may be applied discontinuously at and inside the boundaries of the cruciform attachment pattern, such as in the form of dots, stripes, beads, spirals, etc., and then used to attach the absorbent assembly to the chassis.

The cruciform attachment pattern 210 may be disposed symmetrically with respect to either or both of the longitudinal axis 42 and the lateral axis 44 of the chassis 100. Alternatively, the cruciform attachment pattern 210 may be disposed asymmetrically with respect to either or both of the longitudinal axis 42 and the lateral axis 44. In addition, the cruciform attachment pattern 210 may be disposed symmetrically with respect to either or both of the side edges 237 and the front edge 236 and the back edge 238 of the absorbent assembly 200. Alternatively, the cruciform attachment pattern 210 may be disposed asymmetrically with respect to either or both of the side edges 237 and front edge 236 and back edge 238.

Suitable configurations of cruciform attachment patterns are disclosed in U.S. patent application Ser. No. 10/880,128 filed on 29 Jun. 2004.

The absorbent core 250 may be disposed between a lower covering sheet that is disposed on the exterior face of the absorbent core 250 and an upper covering sheet that is disposed on the interior face of the absorbent core 250. Such an upper covering sheet and lower covering sheet may be attached together to contain the absorbent core 250 between them and thereby form the absorbent assembly 200. For example, in the exemplary absorbent assembly 200 shown in FIG. 25, FIG. 26, and FIG. 27, an upper covering sheet 24 and a lower covering sheet 25 are attached together at or adjacent to the side edges 237 of the absorbent assembly 200 in longitudinally extending attachment zones 29. Alternatively, the upper covering sheet 24 and the lower covering sheet 25 may be attached together in places other than the side edges 237, e.g., at or adjacent to the end edges 236 and 238 of the absorbent assembly 200, or at or adjacent to both the end edges 236 and 238 and the side edges 237. Both the upper covering sheet and the lower covering sheet are water vapor-permeable, i.e., breathable.

The upper covering sheet 24 is water-permeable and allows liquid waste to pass through to the absorbent core 250, where the liquid waste is absorbed. The lower covering sheet 25 may be water-impermeable. However, the lower covering sheet 25 preferably is water-permeable. In embodiments in which both the upper covering sheet 24 and the lower covering sheet 25 are water-permeable, any liquid waste that is deposited onto the upper covering sheet 24 but does not pass through the upper covering sheet 24 to the absorbent core 250 can flow around an edge of the absorbent assembly 200 to reach the lower covering sheet 25 and then pass through the lower covering sheet 25 to the absorbent core 250.

The upper covering sheet 24 may form the interior surface 202 of the absorbent assembly 200 that is intended to be placed against the body of the wearer. The upper covering sheet 24 preferably is formed of a soft material that will not irritate the skin of the wearer. Many materials that are suitable for a water-permeable covering sheet are well-known in the art, including synthetic nonwovens such as spunbonded or carded polypropylene, polyester, or rayon. Likewise, many materials that are suitable for a covering sheet that is water-impermeable are well-known in the art, including the materials that are suitable for the backsheet 26.

In the exemplary absorbent assembly 200 shown in FIG. 25, FIG. 26, and FIG. 27, the upper covering sheet 24 and the lower covering sheet 25 are of the same size, i.e., both the upper covering sheet 24 and the lower covering sheet 25 extend to the front edge 236 and back edge 238, as well as to the side edges 237 of the absorbent assembly 200. Alternatively, the upper covering sheet 24 and the lower covering sheet 25 may differ in size. For example, the upper covering sheet may extend longitudinally only to an extent sufficient to cover the absorbent core and the lower covering sheet may extend longitudinally beyond the upper covering sheet toward or to the adjacent waist edge. Such an extended covering sheet may serve to isolate the skin of the wearer from a portion of the backsheet 26 as may be desirable, for example, when the loincloth diaper 20 is worn under conditions in which contact between the skin and a backsheet film could be uncomfortable.

As another example, the lower covering sheet 25 may be larger than the upper covering sheet 24 and may be wrapped over the side edges 257 of the absorbent core 250 onto the interior surface of the absorbent core 250, where the upper covering sheet 24 and the lower covering sheet 25 may be attached together. Alternatively, in place of a separate upper covering sheet 24 and a separate lower covering sheet 25, a single covering sheet may be wrapped around the absorbent core 250 and attached to itself to contain the absorbent core 250. Such a single covering sheet forms an upper layer and a lower layer when wrapped around the absorbent core 250 and, in general, the description of the separate upper covering sheet 24 and lower covering sheet 25 are intended to apply to such upper and lower layers of a wrapped single covering sheet.

The absorbent core 250 includes a storage component 272 that serves to absorb and retain liquid bodily waste materials. Suitable known materials for the absorbent core storage component include cellulose fibers in the form of comminuted wood pulp, commonly known as “airfelt”, natural or synthetic fibrous materials, and superabsorbent polymers, used either singly or in mixtures and commonly formed into layers or sheets, etc. These absorbent materials may be used separately or in combination. Many known absorbent materials may be used in a discrete form, i.e., in the form of fibers, granules, particles, and the like. Such a discrete form of an absorbent material may be immobilized by an adhesive material that attaches the discrete pieces together to form a coherent layer or that attaches the discrete pieces to a substrate layer or that attaches the discrete pieces both to each other and to the substrate layer.

The absorbent core may include an acquisition component in addition to one or more storage components. The absorbent core acquisition component serves to acquire deposited liquid bodily waste material and transfer it to the absorbent core storage component. Any porous absorbent material which will imbibe and partition liquid bodily waste material to the storage component or components may be used to form the acquisition component. Preferred materials for the acquisition component include synthetic fiber materials, open celled polymeric foam materials, fibrous nonwoven materials, cellulosic nonwoven materials, and various combination synthetic/cellulosic nonwoven materials. For example, the acquisition component may be formed of a nonwoven web or webs of synthetic fibers including polyester, polypropylene, and/or polyethylene, natural fibers including cotton and/or cellulose, blends of such fibers, or any equivalent materials or combinations of materials. Examples of such acquisition materials are more fully described in U.S. Pat. No. 4,950,264 issued to Osborn on Aug. 21, 1990. High loft nonwoven acquisition materials suitable for the acquisition component of the present invention can be obtained from Polymer Group, Inc., (PGI), 450 N.E. Blvd, Landisville, N.J. 08326, U.S.A., under the material code designation of 98920.

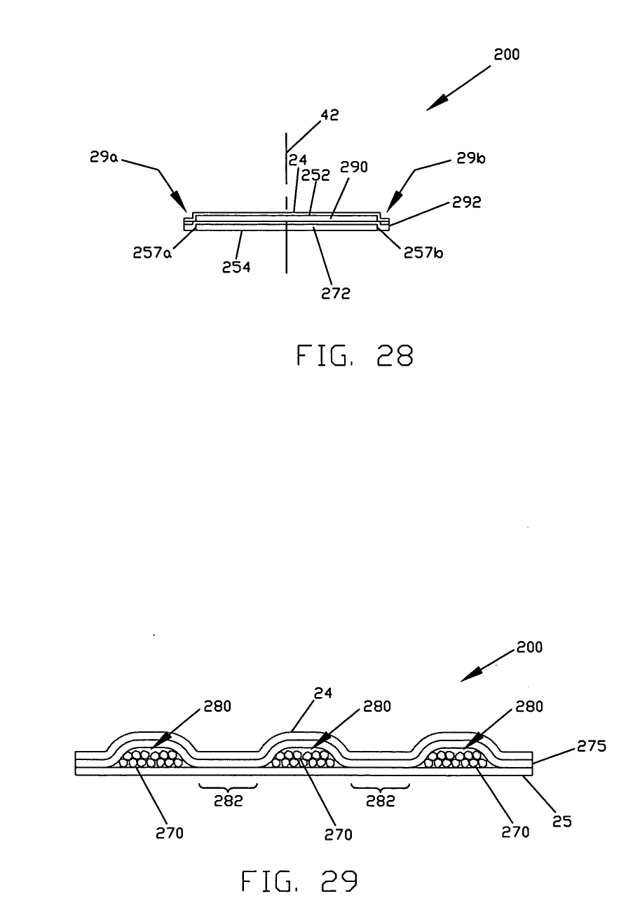

Such an absorbent core acquisition component 290 is shown overlying the absorbent core storage component 272 in FIG. 28. A separation sheet 292 of, e.g., a tissue or a nonwoven material, may be disposed between the absorbent core storage component 272 and the absorbent core acquisition component 290 to help ensure that none of the gel formed by a superabsorbent polymer that may be included in the absorbent core storage component reaches the skin of the wearer. This separation sheet 292 may extend laterally beyond the side edges 257 of the absorbent core 250 and the upper covering sheet 24 may be attached to the separation sheet 292. In this arrangement, the liquid bodily waste material that is deposited onto the upper covering sheet 24 will pass through the thickness of the upper covering sheet 24 to be absorbed by the absorbent core acquisition component 290, and some or all of it may then pass through the thickness of the separation sheet 292 and then be absorbed and retained by the absorbent core storage component 272.

In some exemplary embodiments, an absorbent core storage component may include the discrete form of an absorbent material that is immobilized in pockets formed by a layer of a thermoplastic material, such as a hot melt adhesive material, that intermittently contacts and adheres to a substrate sheet, while diverging away from the substrate sheet at the pockets. Absorbent core components having such structures and being suitable for the storage of liquid bodily wastes are described in U.S. patent applications Ser. Nos. 10/776,839 and 10/776,851, both filed on 11 Feb. 2004 in the name of Ehrnsperger et al. An exemplary absorbent core storage component 272 having such a structure is shown in FIG. 29. In this absorbent core storage component 272, particles 270 of a superabsorbent polymer are contained inside pockets 280 formed by a layer 275 of a thermoplastic material. The absorbent core storage component may include both particles of superabsorbent polymer and airfelt and both materials may be contained inside the pockets formed by the layer of the thermoplastic material. Alternatively, as shown in FIG. 29, an exemplary absorbent core storage component may contain no airfelt and therefore the component can be made relatively thinner and more flexible for the comfort of the wearer. In addition, the particles of the superabsorbent polymer can be immobilized relatively more easily in the absence of airfelt. As shown in FIG. 29, the layer 275 of the thermoplastic material intermittently contacts and adheres to a substrate sheet 274 at the areas of attachment 282. Between the areas of attachment 282, the layer 275 diverges away from the substrate sheet 274 to form the pockets 280. The layer 275 may have the form of a sheet of fibers of the thermoplastic material through which the liquid waste may pass to the particles to be absorbed by the particles 270 of the superabsorbent polymer.

In FIG. 29, a separate thermoplastic layer covering sheet 276 is shown overlying the layer 275 of the thermoplastic material. Alternatively, the separate thermoplastic layer covering sheet 276 may be omitted. As another alternative, two absorbent core storage components each like that shown in FIG. 29 except for the omission of the thermoplastic layer covering sheet 276 may be superposed with one absorbent core storage component inverted such that the respective substrate sheets distally oppose each other. In such a combination of absorbent core storage components, either or both of the distally opposing substrate sheets may serve respectively as either or both of an upper covering sheet and a lower covering sheet for the absorbent assembly. Alternatively, the absorbent assembly may include a separate lower covering sheet and/or a separate upper covering sheet.

Statements of Incorporation by Reference and Intended Scope of Claims

The disclosures of all patents, patent applications and any patents which issue thereon, as well as any corresponding published foreign patent applications, and all publications listed and/or referenced in this description, are hereby incorporated herein by reference. It is expressly not admitted that any of the documents or any combination of the documents incorporated herein by reference teaches or discloses the present invention.

While particular embodiments and/or individual features of the present invention have been described herein, it would be obvious to those skilled in the art that various other changes and modifications can be made without departing from the spirit and scope of the invention. Further, it should be apparent that all combinations of such embodiments and features are possible and can result in preferred executions of the invention. Therefore, the appended claims are intended to cover all such changes and modifications that are within the scope of this invention.

What is claimed is:

1. A disposable loincloth diaper comprising:
an absorbent assembly comprising an absorbent core; and
a chassis having a longitudinal axis, a lateral axis, a front waist region having a front waist edge, a back waist region having a back waist edge, a crotch region between the waist regions, laterally opposing side edges extending between the front waist edge and the back waist edge, an exterior surface, and an interior surface to which the absorbent assembly is attached,
the chassis comprising a water-impermeable backsheet and laterally opposing side flaps attached to the interior surface adjacent to their longitudinally distal ends, each of the side flaps having a longitudinally extending elastic gathering member attached adjacent to its proximal edge,
the chassis further comprising fastening elements disposed on the exterior surface adjacent to the waist edges, the fastening elements being adapted to maintain each of the waist edges in a folded-over configuration forming a laterally extending tunnel such that a portion of the exterior surface is disposed inside the tunnel, the tunnel being adapted to have a support element worn around a waist of a wearer extending through it and used to support the waist regions adjacent to the waist of the wearer and thereby position the absorbent assembly to receive and contain urinary and fecal waste excreted by the wearer.

2. The disposable loincloth diaper of claim 1 further comprising the support element.

3. The disposable loincloth diaper of claim 2 pre-assembled with the support element extending through the tunnel.

4. The disposable loincloth diaper of claim 2 wherein the support element includes an elastic material.

5. The disposable loincloth diaper of claim 2 wherein the support element has the form of a closed loop.

6. The disposable loincloth diaper of claim 1 wherein at least one of the fastening elements is laterally continuous.

7. The disposable loincloth diaper of claim 1 wherein at least one of the fastening elements is laterally discretely segmented.

8. The disposable loincloth diaper of claim 1 wherein at least one of the fastening elements is openable and refastenable.

9. The disposable loincloth diaper of claim 1 wherein at least one of the fastening elements includes an adhesive material.

10. The disposable loincloth diaper of claim 1 wherein at least one of the fastening elements includes a cohesive material.

11. The disposable loincloth diaper of claim 1 wherein at least one of the fastening elements includes a mechanical fastener.

12. The disposable loincloth diaper of claim 1 wherein the absorbent assembly is attached to the chassis in a cruciform attachment pattern.

13. The disposable loincloth diaper of claim 12 wherein at least a portion of the chassis lying outside the cruciform pattern is laterally extensible.

14. The disposable loincloth diaper of claim 1 wherein the backsheet is a laminate of a film and a nonwoven material disposed exteriorly of the film.

15. The disposable loincloth diaper of claim 14 wherein at least one of the fastening elements includes hooks adapted to engage with the nonwoven material of the backsheet.

16. A disposable loincloth diaper comprising:
an absorbent assembly comprising an absorbent core; and
a chassis having a longitudinal axis, a lateral axis, a front waist region having a front waist edge, a back waist region having a back waist edge, a crotch region between the waist regions, laterally opposing side edges extending between the front waist edge and the back waist edge, an exterior surface, and an interior surface to which the absorbent assembly is attached,
the chassis comprising a water-impermeable backsheet and laterally opposing side flaps attached to the interior surface adjacent to their longitudinally distal ends, each of the side flaps having a longitudinally extending elastic gathering member attached adjacent to its proximal edge,
the chassis further comprising fastening elements disposed on the interior surface adjacent to the waist edges, the fastening elements being adapted to maintain each of the waist edges in a folded-over configuration forming a laterally extending tunnel such that a portion of the interior surface is disposed inside the tunnel, the tunnel being adapted to have a support element worn around a waist of a wearer extending through it and used to support the waist regions adjacent to the waist of the wearer and thereby position the absorbent assembly to receive and contain urinary and fecal waste excreted by the wearer.

17. The disposable loincloth diaper of claim 16 wherein the chassis further comprises an inner liner formed of a nonwoven material and at least one of the fastening elements includes hooks adapted to engage with the nonwoven material of the inner liner.

18. A disposable loincloth diaper comprising:
an absorbent assembly comprising an absorbent core; and
a chassis having a longitudinal axis, a lateral axis, a front waist region having a front waist edge, a back waist region having a back waist edge, a crotch region between the waist regions, laterally opposing side edges extending between the front waist edge and the back waist edge, an exterior surface, and an interior surface to which the absorbent assembly is attached,
the chassis comprising a water-impermeable backsheet and laterally opposing side flaps attached to the interior surface adjacent to their longitudinally distal ends, each of the side flaps having a longitudinally extending elastic gathering member attached adjacent to its proximal edge,
the chassis further comprising fastening elements disposed adjacent to the waist edges, the fastening elements being adapted to maintain each of the waist edges in a folded-over configuration forming a laterally extending tunnel adapted to have a support element worn around a waist of a wearer extending through it and used to support the waist regions adjacent to the waist of the wearer and thereby position the absorbent assembly to receive and contain urinary and fecal waste excreted by the wearer.

19. The disposable loincloth diaper of claim 18 wherein the fastening elements are disposed on the exterior surface adjacent to the waist edge in one of the waist regions and on the interior surface adjacent to the waist edge in the opposing waist region, such that a portion of the exterior surface is disposed inside the tunnel in the one of the waist regions and a portion of the interior surface is disposed inside the tunnel in the opposing waist region.

20. The disposable loincloth diaper of claim 18 wherein at least one of the fastening elements is laterally discretely segmented.
